(19) United States
(12) Patent Application Publication
(10) Pub. No.: US 2025/0176805 A1
(43) Pub. Date: Jun. 5, 2025

(54) STEREOSCOPIC ASSEMBLY, SURGICAL MICROSCOPE WITH STEREOSCOPIC ASSEMBLY, AND SURGICAL SET

(71) Applicant: Schölly Fiberoptic GmbH, Denzlingen (DE)

(72) Inventors: Johannes Bourbon, Freiburg (DE); Julian NEHLICH, Freiburg (DE)

(73) Assignee: Schölly Fiberoptic GmbH, Denzlingen (DE)

(21) Appl. No.: 18/841,519

(22) PCT Filed: Feb. 20, 2023
(86) PCT No.: PCT/EP2023/054247

§ 371 Date: Aug. 26, 2024

(30) Foreign Application Priority Data
Mar. 3, 2022 (DE) 10 2022 105 089.0
Mar. 3, 2022 (DE) 10 2022 105 090.4

(51) Int. Cl. A61B 1/00

(52) U.S. Cl. CPC A61B 1/00193

(57) ABSTRACT

A stereoscopic arrangement that includes at least one of each of an objective unit, deflection unit, lens group arrangement and at least one image sensor. In this case, an optical sub-path between the deflection unit and the image sensor should be at an acute angle to a plane spanned by an optical sub-path, running between the objective unit and the deflection unit, of the same optical path and the stereoscopic base. In addition, at least one lens group of a lens group arrangement is intended to be provided between the deflection unit and the image sensor. Also, in a stereoscopic arrangement, at least two optical paths can run in a common plane. Further, a surgical microscope having such a stereoscopic arrangement is provided.

### CROSS-REFERENCE TO RELATED APPLICATIONS

This application is a 371 National Phase of International Application No. PCT/EP2023/054247, filed Feb. 20, 2023, which claims priority from German Patent Application No. 10 2022 105 090.4, filed Mar. 3, 2022 and German Patent Application No. 10 2022 105 089.0, filed Mar. 3, 2022, all of which are incorporated herein by reference as if fully set forth.

### TECHNICAL FIELD

The invention relates to a stereoscopic arrangement that implements a stereoscopic imaging system, having an objective unit, a deflection unit, a lens group arrangement and an image sensor unit. The stereoscopic arrangement may in particular comprise at least one objective unit, at least one deflection unit and at least one image sensor. In this case, the image sensor is arranged downstream of the at least one deflection unit with respect to an optical path beginning at a stereoscopic base.

The invention furthermore relates to a surgical microscope having such a stereoscopic arrangement.

Finally, the invention also relates to a surgical kit that is able to be used in surgical interventions and that comprises a stereoscopic arrangement that is attached to a movable robot arm and is able to be pivoted in space with the aid of the robot arm. The arrangement is in this case preferably designed in accordance with the invention.

### BACKGROUND

Such stereoscopic arrangements, surgical microscopes and surgical kits are used for example in surgical interventions. The patient is in this case observed by such a stereoscopic arrangement or such a microscope. The surgeon stands next to the patient and looks at a monitor on which the stereoscopic image that was recorded, or is currently being recorded live, using the stereoscopic arrangement is shown to them.

However, the space available for such an arrangement in the vicinity of the patient is highly limited since the surgeon's view of the monitor has to be clear, and also the operating space below the stereoscopic arrangement and above the patient has to remain freely accessible. The space able to be taken up by such an arrangement is therefore highly limited.

This is particularly the case when the viewing angle of the stereoscopic arrangement is to be changed. In this case, the arrangement, which is attached to a tripod, may be pivoted about one or more axes. As already mentioned, the arrangement may be attached to a robot arm in a manner able to pivot, for example about two axes. This pivoting may result in the surgeon's view of the monitor being obstructed, in particular when pivoting takes place about an axis running parallel to the distance between the two visual beams, the stereoscopic base.

In systems known to date, it is generally the case that the image beams or optical paths are deflected by a deflection unit. An optical path may be considered to be split into multiple sub-paths by the deflection unit. For instance, one sub-path runs between the stereoscopic base and the deflection unit, and one sub-path runs between the deflection unit and the image sensor unit. In systems known to date, the deflection generally takes place in such a way that the optical paths are deflected out of the plane in which the two first sub-paths run, such that the two second sub-paths (which are used for stereoscopic imaging) lead away from the surgeon. It may be the case here that the sub-paths come into the line of sight from the surgeon to the monitor and thus hinder said surgeon in some situations.

### SUMMARY

The invention is based on the object of providing a stereoscopic arrangement and a surgical microscope having a stereoscopic arrangement, the space taken up by which is reduced in such a way that the abovementioned problems are able to be reduced or completely avoided. In particular, the intention here is for ergonomics to be improved when working with a surgical kit, as described at the outset, during a medical procedure.

In order to achieve the stated object, one or more of the features disclosed herein are provided according to the invention. It is therefore in particular proposed, according to the invention, in order to achieve the stated object in stereoscopic arrangements of the type described at the outset, for a sub-path, running between a deflection unit and an image sensor, of an optical path to be at an acute angle to the plane spanned by the stereoscopic base and a sub-path, running between the objective unit and the deflection unit, of the same optical path, and wherein at least one lens group of a lens group arrangement is arranged between a deflection unit and an image sensor.

The optical path downstream of the deflection unit is therefore able to be guided away to the side of the patient and the operating space. This may also apply to both optical paths. In particular when the stereoscopic arrangement is pivoted, the surgeon's view of the monitor and of the operating space, that is to say for example the operating area currently being observed, is thus able to be kept clear.

Arranging a lens group between the deflection unit and the image sensor likewise saves space between the stereoscopic base and the deflection unit. The first optical sub-path is thus able to be shortened. This results in a compact design of the arrangement in the z-direction (=direction of the optical axis of the objective unit and thus the viewing direction of the stereoscopic arrangement), such that the surgeon always has a clear view of the monitor, even though the arrangement, during use, is arranged between the surgeon's line of sight to the monitor and the operating area under observation.

The lens groups may consist of one or more lenses, and the lenses of a lens group may be in contact with one another, or be designed in a manner spaced apart from one another. By way of example, provision may be made for a respective spacer. A lens group is able to be displaced without changing the distance between the lenses that it contains. By way of example, a lens group may be changeable by virtue of the distance between the lenses that it contains being able to be changed in relation to one another.

As an alternative or in addition, in order to achieve the stated object, according to the invention, provision is made for further features as disclosed herein in a further embodiment, which targets a further stereoscopic arrangement having at least one objective unit, at least one deflection unit, at least one image sensor and at least two optical paths that run from the at least one objective unit, via the at least one deflection unit, to the at least one image sensor. It is therefore in particular proposed, according to the invention, in order to achieve the stated object in stereoscopic arrangements of the type described at the outset, for the optical paths to run in a common plane. In this case, the plane may in particular comprise the stereoscopic base.

The optical paths are thus able to be guided away to the side of the operating area, as a result of which this area, and thus the surgeon's view of the monitor, are able to be kept clear. The optical paths may continue in opposite directions downstream of the deflection unit. However, the optical paths may also run in the same direction and still parallel or at an acute angle to one another downstream of the deflection unit. The optical paths may in this case in particular run parallel to the stereoscopic base.

If for example two optical paths are formed running from the at least one deflection unit to a respective image sensor, then it may be advantageous for these two image sensors to be arranged in a manner offset from one another in the axial direction of the respective optical path. This is because, in this case, waste heat from the image sensors is able to be dissipated better, since the respective image sensors then generate waste heat at two locations, and not at a single location. In addition, there is then more space for establishing contact with the image sensors via flexible printed circuit boards, and also for associated electronic components. The last point, and the fact that the image sensors no longer interfere with one another, also means that the image sensors, more precisely the respective optical paths, are able to move closer to one another. As a result, this makes it possible to obtain a smaller/shorter stereoscopic base.

A shorter stereo base results in principle in a lower stereo impression, which seems to be disadvantageous at first glance. However, the invention has recognized that a smaller stereo base may be advantageous in certain medical applications: This is because, for example, if a narrow body cavity is observed using the stereoscopic arrangement according to the invention (which may in particular be designed as an endoscope), the smaller stereo base results in a small offset of the images. However, especially in the case of a long/deep body cavity, this may cause the front and rear image regions, which are blurred in any case due to the limited depth of field, to be offset to a lesser extent, meaning that it is possible to obtain an image of better overall quality that is easier to process for the human brain. This means that the use characteristics are able to be improved. In addition, the overall arrangement becomes more compact with a smaller stereo base, which in turn offers advantages for the surgeon.

The approach according to the invention of displacing the two image sensors axially in relation to one another/arranging them in a manner offset from one another differs here in particular from conventional concepts, in which a respective eyepiece is provided in place of the image sensor. This is because, if a person is intended to look into an eyepiece using both eyes, it makes no sense whatsoever to move the eyepieces axially in relation to one other.

In order to improve the use characteristics of the arrangement, it is also advantageous to provide a variable aperture stop, preferably in each case in each of the two paths used for stereoscopic vision. This thus makes it possible to arrange at least one adjustable aperture stop downstream of the at least one objective unit in the beam path, using which aperture stop it is possible to adjust a depth of field and/or an optical resolution. If two optical paths are formed running from the at least one deflection unit to a respective image sensor, then it makes sense for a respective adjustable aperture stop to then be arranged in each of these two optical paths. Preferably, the two aperture stops are then able to be arranged in a manner offset from one another in the axial direction, which is particularly advantageous if the associated image sensors, as explained above, are likewise arranged in a manner axially offset from one another. Specifically, this too makes it easier to make the arrangement more compact overall and to form a comparatively small stereo base (which entails the advantages explained above).

If two optical paths are formed running from the at least one deflection unit to a respective image sensor, then two basic designs are also conceivable: Either these two optical paths run in opposite directions, and then preferably also at the same height; or the two optical paths, although they run in the same direction, do so in a manner offset from one another in the direction of a sub-path (in particular the one explained above) running between the objective unit and the at least one deflection unit. In the latter case, the optical paths are thus stacked above one another in relation to the viewing direction of the stereoscopic arrangement (z-direction); in the former case, the paths may, by contrast, run to the left and to the right in relation to the viewing direction. In such embodiments, preference should generally be given, to achieve a compact design transverse to the viewing direction, for the two optical paths, the sub-path running between the objective unit and the at least one deflection unit and the stereoscopic base to run in a common plane.

In one advantageous embodiment, provision may be made for an angle (in particular the abovementioned acute angle) between a sub-path, running between the deflection unit and the image sensor, of an optical path and the plane spanned by the stereoscopic base and a sub-path, running between the objective unit and the deflection unit, of the same optical path to be less than 20°, in particular less than 10° and/or a zero angle. The optical paths may thus, in some cases, also be guided away to the side such that they do not run exactly parallel to the stereoscopic base.

In one advantageous embodiment, provision may be made for a separate objective unit and/or deflection unit and/or at least one lens group of a lens group arrangement and/or a separate image sensor to be provided for each optical path. It is thus easily possible to compensate for differences in optical path lengths by displacing individual lenses or lens groups of the two optical paths in relation to one another. This may also be achieved by displacing individual lens groups of one or both optical paths. By way of example, the components formed in one optical path may be fixed in terms of their position, and the lens groups of the other path may be moved. Using separate lens groups for both paths may advantageously be used to eliminate unfavorable peripheral rays and/or stray light. In addition, it is thereby possible to produce an optimum optical imaging quality for each optical path, because the individual lens groups allow better adjustment of the respective optical path.

In one advantageous embodiment, provision may also be made for a common objective unit and/or deflection unit and/or at least one lens group of a lens group arrangement and/or a common image sensor to be provided for the optical paths. This enables easy installation.

This thus makes it possible, depending on the requirements, in particular depending on the space available for optical components or their desired orientation in space, to provide separate components for each optical path.

However, common components may also be provided for both optical paths: By way of example, a (common) focus unit may be configured upstream of a first deflection unit having common lens groups and/or a zoom lens may be configured downstream of the first deflection unit having separate lens groups for each of the two optical paths. The deflection unit may be configured to be common to both paths and/or separate for each path.

The focus unit may have at least one tunable focus lens by way of which it is possible to change a focus plane of the stereoscopic arrangement. The term “tunable” may be understood here to mean that the refractive power and/or the position of the focus lens is able to be changed along the optical axis. In this case, objects that are located in the current focus plane are imaged sharply onto the at least one image sensor, such that the focus plane forms the object plane of the respective imaging beam path formed by the respective optical path of the stereoscopic arrangement, while the image plane coincides with an active surface of the at least one image sensor. The distance between the focus plane/object plane that is currently set (with the aid of the changeable focus unit) and the at least one objective unit (in particular a first lens surface of this objective unit) may in this case be understood as a working distance. By way of example, if a working distance of 15 cm is set, then objects that are at located at this distance in front of the objective unit are imaged sharply onto the respective image sensor. Different working distances may thus be made possible with the aid of the focus unit (by tuning the focus lens).

Provision may thus be made for at least one focus unit to be formed, which focus unit is arranged upstream of the at least one deflection unit in the beam path and by way of which focus unit it is possible to change a position of a focus plane of the stereoscopic arrangement.

The focus unit may in this case preferably be formed in particular by a lens group and/or in each case as part of the at least one objective unit.

The focus unit is in this case preferably placed such that incident uncollimated imaging beams of the respective optical path leave the focus unit in collimated form and then impinge on the at least one deflection unit as collimated light. Within the stereoscopic arrangement, the focus unit may thus assume the optical function of converting uncollimated incoming light beams (coming from said focus plane/the object under observation) into collimated light.

Such a focus unit may also be designed separately for both optical paths, preference being given to a common focus unit because, in the latter case, it is necessary to drive only one tunable lens, and not to drive two lenses synchronously.

In one advantageous embodiment, provision may be made for the distance between the objective unit and/or the deflection unit and/or the lens groups of a lens group arrangement and/or the image sensor to be the same for each object path. It is thus easy to achieve almost or exactly the same imaging properties of the paths.

In one advantageous embodiment, provision may also be made for the distance between the objective unit and/or the deflection unit and/or the lens groups of a lens group arrangement and/or the image sensor to be different for each optical path. This is particularly expedient in the case of asymmetrical arrangements in which it is necessary to compensate for different optical path lengths.

This means that, depending on the profile of the optical paths, it is possible to select the distances between optical components such that an image of the desired quality is able to be recorded. By way of example, it may be necessary, in the case of identical lengths of the optical paths, to provide different distances between components of the same type for each path. By way of example, it may thus be necessary to provide a different distance between the objective unit and the deflection unit for each path, but to compensate for this by way of a distance between the deflection unit and the image sensor that is also different for each path.

In one advantageous embodiment or in one alternative solution that may possibly have inventive quality on its own, provision may be made for the optical paths to be deflected, by a deflection unit, in the same direction or in opposite directions, and/or to be deflected such that they impinge on image sensors that are arranged on the same side or on opposite sides of an orthogonal plane, which orthogonal plane is oriented orthogonal to the plane spanned by the stereoscopic base and a sub-path, running between the objective unit and the deflection unit, of an optical path and contains a sub-path, running between the objective unit and the deflection unit, of the same optical path.

Thus, depending on the requirements in terms of the geometry of the stereoscopic arrangement, the optical paths may be guided such that they end in the same half-space or in different half-spaces separated by the orthogonal plane.

One preferred application of the invention makes provision for a stereoscopic arrangement according to the invention, in particular as described above, to be used in a surgical microscope.

In order to solve the problem stated at the outset and thus in order to improve the ergonomics and use characteristics of a stereoscopic arrangement according to the invention, the invention proposes a specific surgical kit, that is to say a spatial arrangement of the following components: The surgical kit according to the invention, which may be used in particular in surgical interventions, for instance for observing an operating area on or in the body of a patient, comprises:
a stereoscopic arrangement as as described herein. In this case, the stereoscopic arrangement may be designed for example as part of a surgical microscope or of an endoscope or of an exoscope;
a screen for displaying a stereoscopic image (that is to say in particular for displaying a stereoscopic image data stream, for instance in the form of a live video) that has been recorded or is being recorded with the stereoscopic arrangement. The screen may in this case preferably be arranged behind the stereoscopic arrangement such that a surgeon looking at the screen (in order thus to observe a patient with the aid of the arrangement) has a clear view of the screen. In other words, this thus makes it possible to provide a clear line of sight to the screen for a surgeon, or such a line of sight may be kept clear for a surgeon; preferably, the line of sight in this case runs orthogonal to a display surface of the screen and ends in this display surface.
Finally, the surgical kit comprises a (movable) robot arm (for instance as part of a complex surgical robot), wherein the stereoscopic arrangement is attached to the robot arm and is thus able to be pivoted with the robot arm in space, preferably about two different axes. With the aid of the robot arm, it is thus possible to change a viewing angle of the stereoscopic arrangement. This is because, when the robot arm moves, the stereoscopic arrangement moves as well, wherein a spatial position and/or orientation of an optical axis of the at least one objective unit (and thus the viewing direction of the stereoscopic arrangement) and/or a distance between the at least one objective unit and an object/operating area to be observed (and thus the working distance of the stereoscopic arrangement) are/is then able to be changed.

In order to achieve the object, provision is made, according to the invention, for the stereoscopic base of the arrangement to run parallel to the screen and for the optical paths of the arrangement, that is to say in particular the sub-paths between the at least one deflection unit and the respective image sensor, to be guided away parallel to the stereoscopic base and to the side (with respect to a clear line of sight between the surgeon and the screen).

Such a surgical kit may reduce the burden on the surgeon, especially in the case of difficult microsurgical procedures, wherein the respective carrying along of the stereoscopic arrangement on the robot arm and in particular the changeable focus plane thereof (this being adjustable with the aid of the described focus unit) allows the surgeon to always be shown the desired spatial impression of the current operating area with the aid of the stereoscopic arrangement in different situations with an excellent image sharpness on the screen. With the aid of the described at least one aperture stop, the surgeon is also able here to adapt the resolution and the depth of field of the recorded stereo images as they wish.

This thus makes it possible to implement the stated advantages, in particular with regard to the reduced space taken up by such a stereoscopic arrangement, in a surgical microscope or in an endoscope, and in particular in the above-described surgical kit, and to use them during a medical intervention. It is particularly important to emphasize here that guiding the optical paths away to the side saves valuable installation space in the z-direction (that is to say in the direction of the optical axis of the objective unit of the stereoscopic arrangement), such that said line of sight is kept clear in different positions of the robot arm and the surgeon is thus able to view the full field of view of the stereoscopic arrangement on the screen without obstruction.

Provision may furthermore also be made, in the surgical kit, for two sub-paths of the arrangement that run between the at least one deflection unit and the least one image sensor, with respect to the sub-path, running between the objective unit and the at least one deflection unit, of the stereoscopic arrangement, to be arranged above one another or to run in opposite directions.

As already described in detail, the stereoscopic arrangement according to the invention used in the surgical kit may also comprise a focus unit by way of which it is possible to simultaneously adjust a current position of a focus plane, in particular on the basis of a current position of the robot arm, for two optical paths (which are used for stereoscopic imaging). This in particular makes it possible to achieve a situation whereby the arrangement is able to automatically generate sharp stereo images at different working distances. For this purpose, for example, an autofocus may be implemented in the arrangement with the aid of the focus unit, such that, when the working distance is changed, the system automatically adapts the position of the focus plane to the new working distance.

### BRIEF DESCRIPTION OF THE DRAWINGS

The invention will now be described in more detail with reference to exemplary embodiments, but is not restricted to the exemplary embodiments. Further exemplary embodiments will become apparent by combining the features of one or more claims with one another and/or with one or more features of the exemplary embodiment.

In the figures:

FIG. 1 shows a side view of a stereoscopic arrangement according to the invention,

FIG. 2 shows a frontal view of the stereoscopic arrangement from FIG. 1,

FIG. 3 shows a side view of a further stereoscopic arrangement according to the invention,

FIG. 4 shows a frontal view of the stereoscopic arrangement from FIG. 3,

FIG. 5 shows a side view of a further stereoscopic arrangement according to the invention,

FIG. 6 shows a frontal view of the stereoscopic arrangement from FIG. 5,

FIG. 7 shows a side view of a further stereoscopic arrangement according to the invention,

FIG. 8 shows a frontal view of the stereoscopic arrangement from FIG. 7,

FIG. 9 shows a side view of a further stereoscopic arrangement according to the invention,

FIG. 10 shows a frontal view of the stereoscopic arrangement from FIG. 9,

FIG. 11 shows a side view of a further stereoscopic arrangement according to the invention,

FIG. 12 shows a frontal view of the stereoscopic arrangement from FIG. 11,

FIG. 13 shows a top view of a surgical microscope according to the invention, as part of a surgical kit according to the invention,

FIG. 14 shows a side view of a further stereoscopic arrangement according to the invention,

FIG. 15 shows a frontal view of the stereoscopic arrangement from FIG. 14,

FIG. 16 shows a side view of a further stereoscopic arrangement according to the invention, and

FIG. 17 shows a frontal view of the stereoscopic arrangement from FIG. 16.

### DETAILED DESCRIPTION

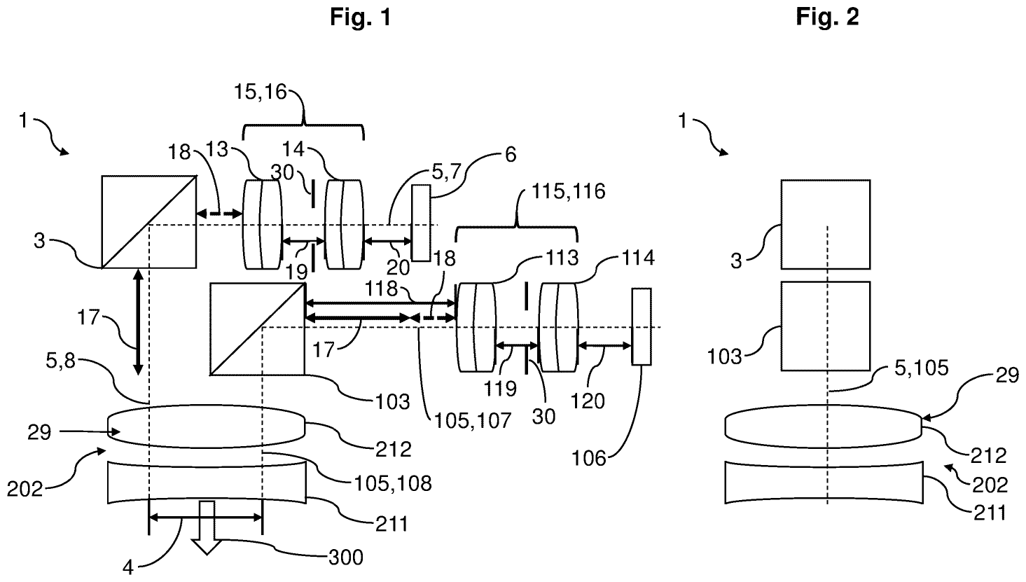

FIG. 1 shows a side view of a stereoscopic arrangement 1 having a common objective unit 202, two deflection units 3, 103 and two optical paths 5, 105, beginning at the stereoscopic base 4 and running via a respective deflection unit 3, 103 to a respective image sensor 6, 106. Each deflection unit 3, 103 splits the respective optical path 5, 105 into two respective sub-paths 7, 107 and 8, 108. The sub-paths 7, 107 run between the deflection unit 3, 103 and the image sensor 6, 106, and the sub-paths 8, 108 run between the stereoscopic base 4 and the deflection unit 3, 103.

The sub-paths 7, 107 are each at an angle to the plane spanned by the sub-path 8, 108 belonging to the same optical path 5, 105 and the stereoscopic base 4, wherein the angle, in this exemplary embodiment, is a zero angle in each case. The objective unit 202 consists of common lens groups 211, 212. Further lens groups 13, 113, 14, 114 of a respective lens group arrangement 15, 115 are located between the deflection unit 3, 103 and the image sensor 6, 106.

The viewing direction of the stereoscopic arrangement 1 is in this case exactly opposite the direction of the respective first sub-path 5, 105; in other words, the arrangement 1 may receive imaging beams from a spatial direction that corresponds to the direction of the respective sub-path 5, 105 and thus record a corresponding image in the viewing direction.

The lens group arrangement 15, 115 may also be a zoom lens 16, 116. The deflection unit 3, 103 may also be arranged within the lens group arrangement 15, 115; for example, there may be a lens group 13, 113 in the sub-path 8, 108 and a lens group 14, 114 in the sub-path 7, 107. It may also be seen in the illustrated exemplary embodiment that an offset 17 between the deflection units 3, 103 leads to an extension of the sub-path 8 in relation to the sub-path 108, and the sub-path 107 is extended in relation to the sub-path 7 by the offset 17 in order to compensate for this. The distance 118 between the deflection unit 103 and the lens group 113 results from the offset 17 and the distance 18 between the deflection unit 3 and the lens group 13. In this exemplary embodiment, for example, the distances 18, 118 are different from one another, whereas the distances 19, 119 between the lens groups 13, 113 and 14, 114 and the distances 20, 120 between the lens groups 14, 114 and the image sensors 6, 106 are the same in each case.

It may also be seen clearly in the exemplary embodiments in the figures that an adjustable aperture stop 30 may be provided at different positions within the respective beam path in order to make it possible to adjust the depth of field. As may be seen in the figures, a respective adjustable aperture stop 30 may preferably be arranged in the respective beam path used for stereoscopic imaging. Preferably, the changeable aperture stop 30 is in this case placed downstream of the lens 212, preferably downstream of the respective first beam splitter 3/103, in the beam path. By way of example, the respective aperture stop 30 may be arranged at the distance 18/118 or at the distance 19/119. These aperture stops 30 are also driven synchronously. By way of example, it may also be seen clearly in FIG. 1 that the two aperture stops 30 there (like the two image sensors 6, 106) are arranged in a manner axially offset from one other, specifically in relation to the respective parallel sub-path 7, 107.

FIG. 2 shows a frontal view of the stereoscopic arrangement 1 from FIG. 1. It is possible here to see the common objective unit 202, consisting of common lens groups 211 and 212, and the deflection units 3 and 103. The optical paths 5 and 105 are largely congruent in the illustrated perspective, since the angle between the sub-path 7, 107, running between the deflection unit 3 and the image sensor 6, 106, of the optical path 5, 105 and the plane spanned by the stereoscopic base 4 and the sub-path 8, 108, running between the objective unit 202 and the deflection unit 3, 103, of the same optical path 5, 105 is a zero angle in each case.

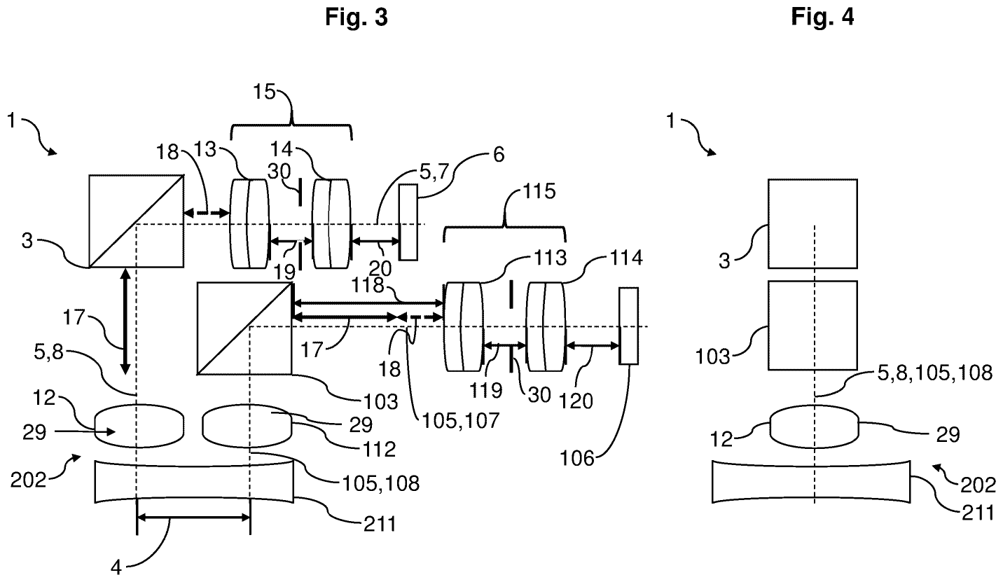

FIG. 3 shows a further stereoscopic arrangement 1 according to the invention, which differs from the arrangement 1 shown in FIG. 1 in that, instead of a common lens group 212 of a common objective unit 202, a separate lens group 12, 112 is provided for each sub-path 8, 108. The objective unit 202, as in FIG. 1, also comprises a common lens group 211.

FIG. 4 shows a frontal view of the stereoscopic arrangement 1 from FIG. 3, having deflection units 3, 103, and the objective unit 202, wherein the lens group 12 belonging to the sub-path 8 and the common lens group 211 are visible.

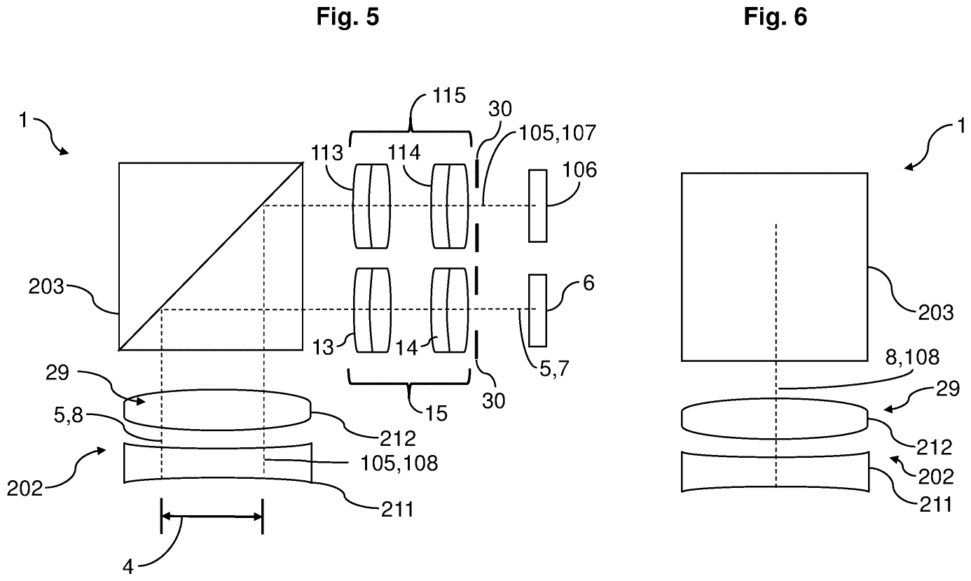

FIG. 5 shows a further stereoscopic arrangement 1 according to the invention, having a common objective unit 202 consisting of common lens groups 211 and 212 and a common deflection unit 203. The optical paths 5, 105 are in this case guided such that the different length of the sub-paths 8 and 108 up to the lens group 13, 113 of the respective lens group arrangement 15, 115 is compensated for. The respective lens groups 13, 113 and 14, 114, as well as the image sensors 6, 106 of the sub-paths 7, 107, thus end up being located next to one another.

FIG. 6 shows a frontal view of the stereoscopic arrangement 1 from FIG. 5, wherein the common lens groups 211 and 212 of the common objective unit 202 and the common deflection unit 203 are visible. The profile of the sub-paths 8 and 108, which is largely congruent from this perspective, is indicated.

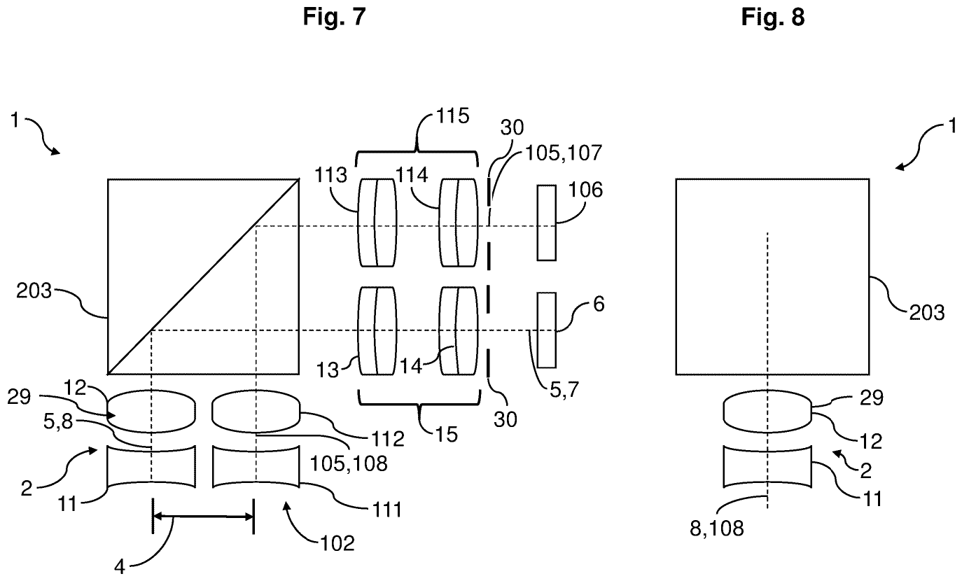

FIG. 7 shows a further stereoscopic arrangement 1 according to the invention, which differs from the arrangement 1 shown in FIG. 5 in that, here, a separate objective unit 2, 102 having two respective lens groups 11, 111 and 12, 112 is provided for each sub-path 8, 108.

FIG. 8 shows a frontal view of the stereoscopic arrangement 1 from FIG. 7, wherein the common deflection unit 203 and the lens groups 11 and 12 of the objective unit 2 belonging to the sub-path 8 are visible. The profile of the sub-paths 8 and 108, which is largely congruent from this perspective, is indicated.

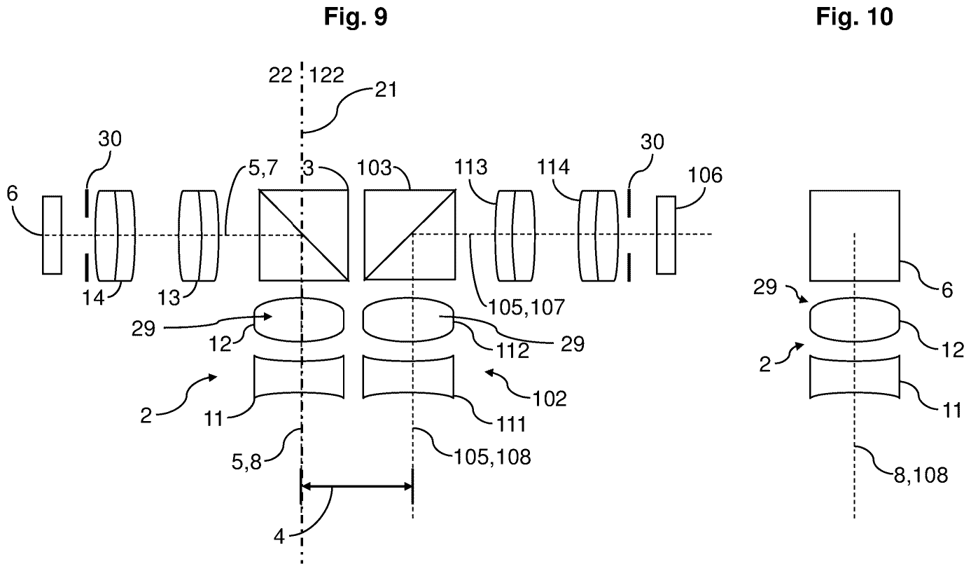

FIG. 9 shows a further stereoscopic arrangement 1 according to the invention, wherein the sub-paths 7 and 107 run in opposite directions from the respective deflection units 3 and 103. In this case, in each case a separate objective unit 2, 102 having lens groups 11, 111 and 12, 112, separate lens group arrangements 15, 115 having lens groups 13, 113 and 14, 114 and separate image sensors 6, 106 are provided for each optical path 5, 105. The projection of the orthogonal plane 21 is also illustrated, wherein the optical paths 5 and 105, in this exemplary embodiment, are deflected by the deflection units 3 and 103 into different half-spaces 22, 122 that are separated by the orthogonal plane 21.

FIG. 10 shows a frontal view of the stereoscopic arrangement 1 from FIG. 9, wherein the objective unit 2 having the lens groups 11 and 12 and the image sensor 6 are visible. The profile of the sub-paths 8 and 108, which are congruent from this perspective, is indicated.

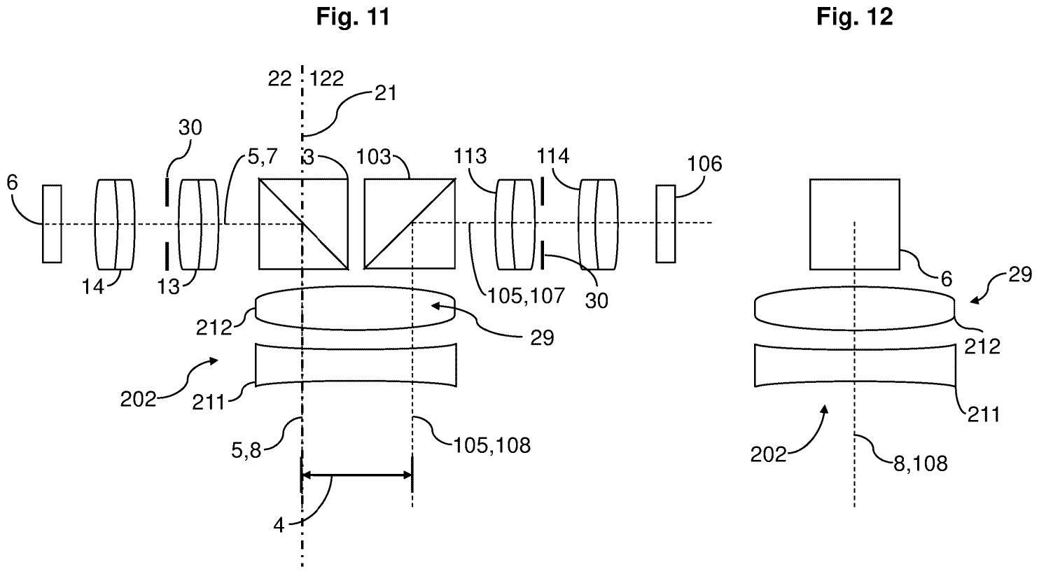

FIG. 11 shows a further stereoscopic arrangement 1 according to the invention, which differs from the arrangement 1 shown in FIG. 9 in that, here, a common objective unit 202 having common lens groups 211 and 212 is provided.

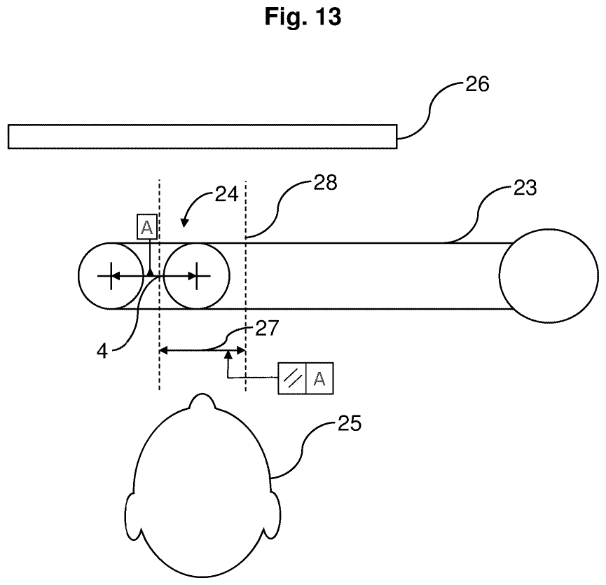

FIG. 12 shows a frontal view of the stereoscopic arrangement 1 from FIG. 11, wherein the common objective unit 202 having the lens groups 211 and 212 and the image sensor 6 are visible. The profile of the sub-paths 8 and 108, which are congruent from this perspective, is indicated.

As mentioned at the outset, in systems known to date, the two optical paths used for stereoscopic vision were typically deflected out of the plane defined by the two first sub-paths (running between the stereoscopic base and the first deflection unit). The invention now proposes, in accordance with the surgical kit according to the invention as already explained above, an alternative solution in which the sub-paths in said plane are guided away to the side (either in one direction, for instance as in the examples of FIGS. 1 to 8, or in two respectively opposite directions, as illustrated in FIGS. 9 to 12). This will be explained by way of illustration below with reference to FIG. 13:

FIG. 13 shows a top view of a surgical microscope 24 according to the invention, mounted on a robot arm 23 and having a stereoscopic arrangement 1, with a surgeon 25 and a monitor in the form of a screen 26. The screen 26 may be used to display a live video image data stream that is currently being recorded by the arrangement 1. The robot arm 23 may in this case be adjusted in space, such that the surgical microscope 24, and thus the arrangement 1, is able to be positioned at different working distances from the patient, more precisely from the operating area to be observed. For this purpose, for example, provision may be made for the robot arm 23 (and with it the arrangement 1) to be able to be pivoted about at least two different axes.

In order always to obtain a sharp image even at different viewing angles and/or different working distances (=distance between the objective unit 2 and the object under observation), the system has an autofocus, which is implemented with the aid of a focus unit of the stereoscopic arrangement 1.

The length of the stereoscopic base 4 of the stereoscopic arrangement 1 corresponds in this case to the inter-eye distance 27 of the surgeon 25, that is to say the lateral distance between the two optical paths 8, 108 is selected according to a typical average value for the inter-eye distance 27 (see for instance FIG. 1 or FIG. 9). In the view of FIG. 13, the robot arm 23 in this case conceals the guidance of the optical paths 5, 105 parallel to the stereoscopic base 4 and to the side away from the line of sight 28 between the surgeon 25 and the screen 26. In other words, those optical paths 7, 107 that run between the respective deflection element 3/103 and the respective image sensor 6/106 are thus guided away in the lateral direction, specifically exactly in the plane that is defined by the two paths 8, 108 and in which plane the stereoscopic base 4 is also located (cf. FIG. 1 or for example FIG. 9). The invention has specifically recognized that this installation space may be used to arrange the imaging optical elements 13, 14/113/114 without the clear view for the surgeon 25 of the screen 26 being obstructed.

FIG. 14 shows a further stereoscopic arrangement 1 according to the invention, which differs from the arrangement 1 shown in FIG. 5 in that, here, a common image sensor 206 is provided.

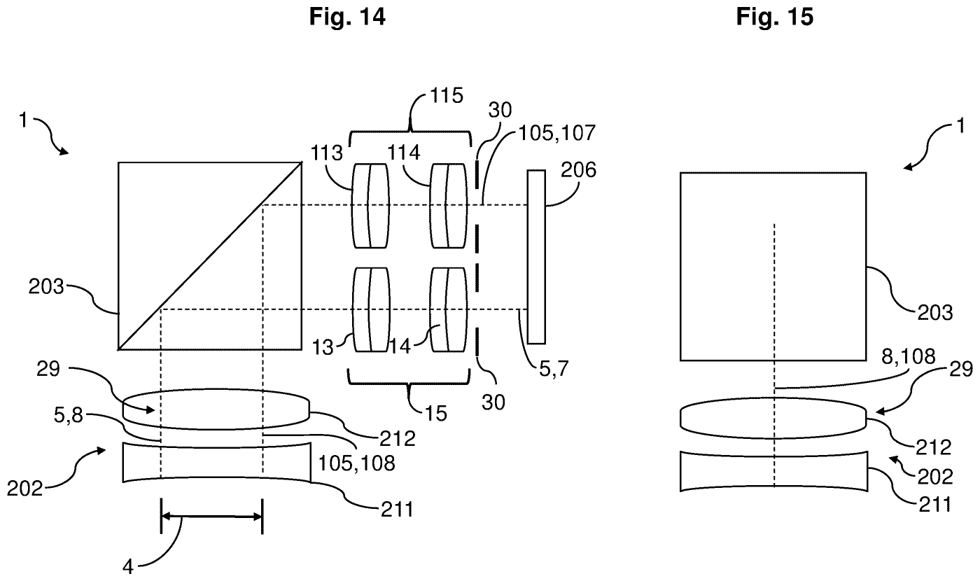

FIG. 15 shows a frontal view of the stereoscopic arrangement 1 from FIG. 14, wherein the common lens groups 211 and 212 of the common objective unit 202 and the common deflection unit 203 are visible. The profile of the sub-paths 8 and 108, which is largely congruent from this perspective, is indicated.

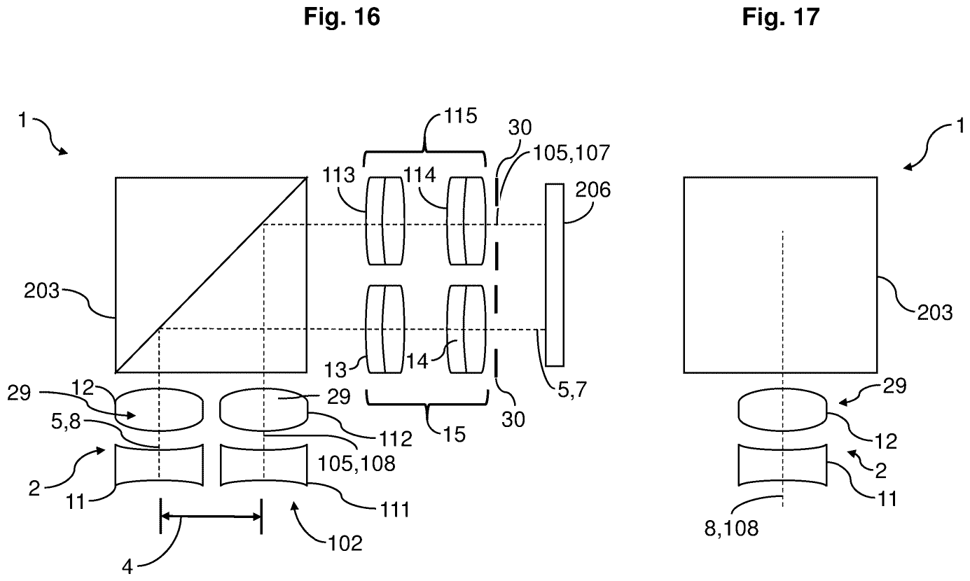

FIG. 16 shows a further stereoscopic arrangement 1 according to the invention, which differs from the arrangement 1 shown in FIG. 7 in that a common image sensor 206 is provided.

FIG. 17 shows a frontal view of the stereoscopic arrangement 1 from FIG. 16, wherein the common deflection unit 203 and the lens groups 11 and 12 of the objective unit 2 belonging to the sub-path 8 are visible. The profile of the sub-paths 8 and 108, which is largely congruent from this perspective, is indicated.

### LIST OF REFERENCE SIGNS

1 stereoscopic arrangement
2 objective unit
3 deflection unit
4 stereoscopic base
5 optical path
6 image sensor
7 sub-path
8 sub-path
11 lens group
12 lens group
13 lens group
14 lens group
15 lens group arrangement
16 zoom lens
17 offset
18 distance
19 distance
20 distance
21 orthogonal plane
22 half-space
23 robot arm
24 surgical microscope
25 surgeon
26 screen
27 inter-eye distance
28 line of sight
29 focus unit
30 aperture stop (for adjusting depth of field and optical resolution)
102 objective unit
103 deflection unit
105 optical path
106 image sensor
107 sub-path
108 sub-path
111 lens group
112 lens group
113 lens group
114 lens group
115 lens group arrangement
116 zoom lens
118 distance
119 distance
120 distance
122 half-space
202 common objective unit
203 common deflection unit
206 common image sensor
211 common lens group
212 common lens group
300 viewing direction (parallel to the optical z-axis of 2/102/202)

1. A stereoscopic arrangement (1), comprising:
at least one objective unit (2, 102, 202),
at least one deflection unit (3, 103, 203
at least one image sensor (6, 106), which is arranged downstream of the at least one deflection unit (3, 103, 203) with respect to an optical path (5, 105) beginning at a stereoscopic base (4),
an optical path (5, 105) having a first sub-path (7, 107) and a second sub-path (8, 108), the first sub-path extends between the at least one deflection unit (3, 103, 203) and the at least one image sensor (6, 106), with the first sub-path (7, 107) being at an acute angle to a plane spanned by the stereoscopic base (4) and the second sub-path (8, 108), which extends between the at least one objective unit (2, 102, 202) and the at least one deflection unit (3, 103, 203), and
in that at least one lens group (13, 14, 113, 114) of a lens group arrangement (15, 115) arranged between the at least one deflection unit (3, 103, 203) and the at least one image sensor (6, 106).

2. A stereoscopic arrangement (1), comprising:
at least one objective unit (2, 102, 202),
at least one deflection unit (3, 103, 203),
at least one image sensor (6, 106), which is arranged downstream of the at least one deflection unit (3, 103, 203) with respect to an optical path (5, 105) beginning at a stereoscopic base (4),
at least two optical paths (5, 105) that extend from the at least one objective unit (2, 102, 202), via the at least one deflection unit (3, 103, 203), to the at least one image sensor (6, 106), and
the optical paths (5, 105) run in a common plane.

3. The stereoscopic arrangement (1) as claimed in claim 2, wherein that at least one image sensor includes two of the image sensors, and two of the optical paths (7, 107) are formed that extend from the at least one deflection unit (3, 103, 203) to a respective one of the image sensors (6, 106), and wherein the two image sensors (6, 106) are arranged offset from one another in an axial direction of the respective optical path (7, 107).

4. The stereoscopic arrangement (1) as claimed in claim 2, further comprising at least one adjustable aperture stop (30) arranged downstream of the at least one objective unit (2, 102, 202) in the beam path, said aperture stop being configured to adjust a depth of field and/or an optical resolution.

5. The stereoscopic arrangement (1) as claimed in claim 2, wherein two of the optical paths (7, 107) are formed that extend from the at least one deflection unit (3, 103, 203) to a respective one of the image sensors (6, 106), and wherein the two optical paths (7, 107) extend
in opposite directions, or
in a same direction, but offset from one another in a direction of a sub-path (8, 108) that extends between the objective unit (2, 102, 202) and the at least one deflection unit (3, 103, 203).

6. The stereoscopic arrangement (1) as claimed in claim 1, further comprising at least one focus unit (29) arranged upstream of the at least one deflection unit (3, 103, 203) in the beam path and the focus unit being configured to change a position of a focus plane of the stereoscopic arrangement (1).

7. The stereoscopic arrangement (1) as claimed in claim 1, wherein the acute angle between the first sub-path (7, 107), which extends between the deflection unit (3, 103, 203) and the image sensor (6, 106), of the optical path (5, 105) and the plane spanned by the stereoscopic base (4) and the second sub-path (8, 108), which extends between the objective unit (2, 102, 202) and the deflection unit (3, 103, 203), of the same optical path (5, 105) is less than 20°.

8. The stereoscopic arrangement (1) as claimed in claim 2, wherein at least one of a separate one of the objective units (2, 102), a separate one of the deflection units (3, 103), a separate one of the at least one lens group (13, 14, 113, 114) of the lens group arrangement (15, 115), or a separate one of the image sensors (6, 106) is provided for each said optical path (5, 105).

9. The stereoscopic arrangement (1) as claimed in claim 1, wherein at least one of a common one of the at least one objective unit (202), a common one of the at least on deflection unit (203), a common one of the at least one lens group (13, 14, 113, 114) of the lens group arrangement (15, 115), or a common one of the at least one image sensor (6, 106) is provided for the optical paths (5, 105).

10. The stereoscopic arrangement (1) as claimed in claim 2, wherein a distance between at least one of the at least one objective unit (2, 102, 202), the at least one deflection unit (3, 103, 203), lens groups (13, 14, 113, 114) of a lens group arrangement (15, 115), [and/] or the at least one image sensor (6, 106) is the same for each said optical path (5, 105).

11. The stereoscopic arrangement (1) as claimed in claim 2, wherein a distance between at least one of the at least one objective unit (2, 102, 202), the at least one deflection unit (3, 103, 203), lens groups (13, 14, 113, 114) of a lens group arrangement (15, 115), or the at least one image sensor (6, 106) is different for each said optical path (5, 105).

12. The stereoscopic arrangement (1) as claimed in claim 2, wherein the optical paths (5, 105) are deflected, by the at least one deflection unit (3, 103, 203),
in the same direction or
in opposite directions.

13. A surgical microscope (24) comprising:
the stereoscopic arrangement (1) as claimed in claim 1.

14. A surgical kit for use in surgical interventions, comprising:
the stereoscopic arrangement (1) as claimed in claim 1 designed as part of a surgical microscope (24) or of an endoscope,
a screen (26) for displaying a stereoscopic image that has been or is being recorded by the stereoscopic arrangement (1),
a movable robot arm (23), wherein the stereoscopic arrangement (1) is attached to the robot arm (23) in a manner able to pivot such that a viewing angle of the stereoscopic arrangement (1) is able to be changed, and the stereoscopic arrangement (1) is oriented such
that the stereoscopic base (4) of the arrangement (1) runs parallel to the screen (26) and
the optical paths (5, 105) of the stereoscopic arrangement (1) are guided away parallel to the stereoscopic base (4) and to a side with respect to a clear line of sight (28) between the surgeon (25) and the screen (26).

15. The surgical kit as claimed in claim 14 [[10]], wherein two of the first sub-paths (7, 107) of the stereoscopic arrangement (1) which extend between the at least one deflection unit (3, 103, 203) and the at least one image sensor (6, 106), with respect to the second sub-path (8, 108), which extends between the objective unit (2, 102, 202) and the at least one deflection unit (3, 103, 203), of the stereoscopic arrangement (1),
are arranged above one another or
run in opposite directions.

16. The surgical kit as claimed in claim 14, wherein the stereoscopic arrangement (1) comprises a, focus unit (29) configured for simultaneous adjustment of a current position of a focus plane (30) for two of the optical paths (5, 105).

17. The stereoscopic arrangement (1) as claimed in claim 2, wherein
the two optical paths (7, 107) are formed that extend from the at least one deflection unit (3, 103, 203) to a respective one of the image sensors (6, 106), and
a respective adjustable aperture stop (30) is arranged in each of the two optical paths (7, 107), and the two aperture stops (30) are arranged offset from one another in an axial direction.

18. The stereoscopic arrangement (1) as claimed in claim 6, wherein
incident uncollimated imaging beams of the respective optical path (5, 105) leave the focus unit (29) in collimated form and then impinge on the at least one deflection unit (3, 103, 203) as collimated light.

19. The stereoscopic arrangement (1) as claimed in claim 2, wherein
the optical paths (5, 105) are deflected by the at least one deflection unit (3, 103, 203) such that they impinge on the at least one image sensor (6, 106) that are arranged on the same side or on opposite sides of an orthogonal plane (21) that is oriented orthogonal to a plane spanned by the stereoscopic base (4) and a sub-path (8, 108), running between the objective unit (2, 102, 202) and the deflection unit (3, 103, 203), of the optical path (5, 105), and the orthogonal plane (21) contains a sub-path (8, 108), running between the objective unit (2, 102, 202) and the deflection unit (3, 103, 203), of the same optical path (5, 105).
